(19) United States
(12) Patent Application Publication
(10) Pub. No.: US 2026/0069182 A1
(43) Pub. Date: Mar. 12, 2026

(54) VISUAL DISTRACTION DETECTION METHOD IN DRIVING MONITORING SYSTEM

(71) Applicant: XYLON d.o.o., Zagreb (HR)

(72) Inventors: Predrag VIDAS, Zagreb (HR); Viseslav CELAN, Imotski (HR)

(73) Assignee: XYLON d.o.o., Zagreb (HR)

(21) Appl. No.: 19/259,300

(22) Filed: Jul. 3, 2025

Related U.S. Application Data
(63) Continuation of application No. PCT/EP2023/050150, filed on Jan. 5, 2023.

(51) Int. Cl. A61B 5/18; A61B 5/00; A61B 5/16; B60Q 9/00; B60R 16/023; B60T 7/12; G06V 10/764; G06V 20/59; G06V 40/16; G06V 40/18; G06V 40/20; G07C 5/04

(52) U.S. Cl. CPC A61B 5/18; A61B 5/0077; A61B 5/163; A61B 5/6893; B60Q 9/00; B60R 16/0231; B60T 7/12; G06V 10/764; G06V 20/597; G06V 40/20; G07C 5/04; G06V 40/171; G06V 40/193

(57) ABSTRACT

A system and method of operation of a visual distraction detection module for use within a driving monitoring system or implemented within a driving monitoring method, which can implement: a driver's gaze zone estimation data for each captured frame where the gaze zones can be previously defined for the specific vehicle, and vehicle info captured from the vehicle. The system or method can perform scoring of a plurality of time windows by a value ranging from 0 to 1, based on the potential usefulness level (PUL) of the gaze zone recognized for each frame, then, can create a data vector, and can extract the possible driver's secondary task. The system and method can estimate the instant driver's visual distraction level as a discrete output {0, 1, 2, 3}, where the gaze zones can be re-classified for each captured frame, and the zones' PUL table can be dynamically updated.

### CROSS-REFERENCE TO RELATED APPLICATION(S)

The present application is a continuation of International App. No. PCT/EP2023/050150 filed Jan. 5, 2023, the entire content and disclosure of which is hereby incorporated by reference in entirety.

### FIELD

The present disclosure relates to or can involve a visual distraction detection method for a driving monitoring system.

### SUMMARY

A method of operation of a visual distraction detection module can be provided or implemented within a driving monitoring system that can provide the visual distraction detection module with the following data:
a driver's gaze zone estimation data for each captured frame by one or more cameras situated within the vehicle oriented toward the driver, where the gaze zones are previously defined for the specific vehicle with the provision that one or more zones change their function in time, and where each of the zones is dynamically classified for each recorded frame into primary, secondary, or tertiary gaze zone based on its purpose in current driving conditions, and
vehicle info, captured from the vehicle systems, which at least comprises data regarding the speed, the steering angle, and in some cases, the turn signal.

The method of operation can comprise the following steps for each newly captured frame:
A. performs the scoring of a plurality of time windows by a value ranging from 0 to 1 and calculates the driver's gaze zones usage distribution within the primary, secondary, and tertiary gaze zones for each selected time window of the plurality of time windows,
   where at least one of the time windows lasts between 2-12 seconds, and where each of the time windows is sliced in subintervals defined by time width of captured camera frames,
   where the scoring for each of the time windows is calculated by averaging scores of all captured frames within the time window, where the score for each captured frame is its potential usefulness level (PUL) value, retrieved from the PUL table, assigned to the vehicle zone to which is recorded the driver's gaze zone estimation for the current frame,
B. create a data vector that consists or is comprised of primary, secondary, and tertiary gaze zone usage distribution and corresponding scoring data performed for predefined time windows,
C. extracts the driver's mental focus and signals with TRUE if the driver is involved in any detectable secondary task by using the data from step B and performing the estimation via a previously trained AI algorithm or using an analytical expression resulting in the TRUE/FALSE output,
D. estimates the instant driver's visual distraction level (VDL) resulting in the discrete output {0, 1, 2, 3} and optionally activating the alarm, where the estimation is performed by a visual distraction expert algorithm which combines the data from step C, the vehicle info, and the instant gaze zone estimation for the current frame.

Optionally, the method (or operation or functionality of the system) may be characterized by the fact that the gaze zones can be re-classified for each captured frame in a way that the estimated driver's gaze zone can be assigned as:
primary gaze zone, if it serves as the gaze zone for keeping the vehicle trajectory,
secondary gaze zone if it serves for planning the driving, and
tertiary gaze zone for all other gaze zones.

Furthermore, the PUL table for all gaze zones can be updated for each frame in a way that:
(i) for the gaze zone detected in the current frame as the primary zone its PUL value is set to 1,
(ii) for the gaze zone detected in the current frame as the secondary or tertiary zone, its PUL value is decreased, and
(iii) the PUL values of other gaze zones, that are not detected in the current frame, are potentially modified if they are correlated with the gaze zone detected in the current frame.

According to one or more embodiments, all new PUL values may stay (e.g., may be required to stay) within the interval [0,1] and can be stored in the PUL table together with their increase and decrease rates defined by the scoring model in step A.

According to one or more embodiments, the PUL values of other gaze zones that are not detected in the current frame, can be currently increased, decreased, or remain unaffected, which can depend on the contribution of each particular zone to the driver's visual distraction if, in the next frame, this zone is estimated as the driver's gaze zone. The PUL decrease or increase rate for any gaze zone can be a unique function of time, the zone, its currently recorded PUL value, and predefined interaction mode with other potential gaze zones.

According to one or more embodiments, for the method of operation, the VD level can be set to 1 if step C detected a secondary task performed by the driver. Moreover, the VD level can be raised to 2 and can trigger the alarm if the speed is above the lower threshold speed, and
(i) for speeds higher than the upper threshold speed, the alarm can be activated the moment, i.e., the frame after the level of visual distraction is at level 1, or
(ii) for speeds between the lower threshold speed and the upper threshold speed, the alarm can be activated within the delay, depending on the secondary task intensity which can be calculated as a percentage of the TRUE state within the specified time window.

According to one or more embodiments, for the method of operation, the duration of the current gaze can be evaluated. If the current gaze is directed in the tertiary zone for a time greater than the defined time interval, the VD level can be raised to 2, for the following conditions:
(i) for speeds that are lower than the lower threshold speed, the defined time interval can be Thi,
(ii) for speeds between the lower threshold speed and the upper threshold speed, the defined time interval can be linearly decreased from Thi to Tlow, and
(iii) for speeds above the upper threshold speed, the defined time interval is set to Tlow;
where the time interval Thi is greater than Tlow time interval.

Optionally, the lower threshold speed can be set to be 10 km/h, the higher threshold speed can be set to be 60 km/h, while Thi can be set to 2.5 sec and Tlow to 2.0 sec.

The duration of the current gaze can be evaluated, if the current gaze is not directed in the primary zone for the predefined time interval, for instance, 3-6 sec, more preferably 4 sec, the VD level can be raised to 2.

The VD level can be set to 3 if the VD level is maintained on 2 for the time interval 0.5-5 sec, more preferably 1-3 sec, where the time interval can be selected to be inversely proportional to the vehicle speed.

The output to the driver, for instance, an alarm, for prompting the driver, can be selected from one or more signals from the group consisting of or comprising visual alarms, audio alarms, automatic braking, shaking a steering wheel, and/or turning on/off different devices in the vehicle cabin. Optionally, a signal can be sent to other vehicle driver safety systems.

Some or all alarms are turned off or suspended if the driver's gaze is directed to the primary gaze zone longer than the predefined time interval.

### DESCRIPTION OF FIGURES

FIG. 1 depicts a driving monitoring system with a visual distraction detection (VDD) module or circuitry according to one or more embodiments of the present disclosure.

FIG. 2 shows portions of the visual distraction detection module.

FIG. 3 shows module(s) that can update each zone's importance, regarding the current driver's behavior and which can store the data within the PUL table.

FIG. 4, situations A-F, depicts scenarios regarding the decrease/increase rates for a particular zone, vs. the current PUL value of the same zone, while the value is between 0 to 1.

FIGS. 5A-5E show time-dependent behaviour of some particular gaze zone PUL value vs. time. Curves a, b and c show particular choices of the increase/decrease rates, some of them correlated with the examples from FIG. 4.

FIG. 6 shows an example of the vehicle's interior divided into exemplary zones.

FIG. 7 shows estimated driver's gaze zones vs. time for zones enumerated in FIG. 6 as (51, 52, 53, 54, 55), and for the same set of zones their PUL values vs. time, for the same time instances.

FIG. 8 shows an illustrative example where only two zones are detected within the time window, e.g., zones (51, 54). The figure depicts the corresponding PUL values in time, current frame score, recovery signal (RCVY) which can be used for alarm deactivation, 2^{nd} task detection, and finally the VDL output {0, 1, 2, 3} that can trigger the alarm in some instances.

### DETAILED DESCRIPTION

There is ongoing research related to driver fatigue, distraction, workload, and other driver-state related factors creating potentially dangerous driving situations. This is not surprising considering that approximately ninety-five percent of all traffic incidents are due to driver error, of which, driver inattention may be regarded as the most common causative factor.

Knowing where a driver is looking is generally accepted as an important input factor for systems designed to avoid incidents, and in particular, crashes. Drivers are often unaware of the effects that drowsiness and distraction have on their own abilities for vehicle control. Humans in general, and particularly as drivers, are poor judges of their own performance capabilities. Typically, a driver's self-impression of his or her capabilities is better than actuality. Even persons who have basically good driving skills, will not always perform at the same skill level while driving. Furthermore, there are many times during driving when very little demand is placed on the driver with respect to the execution of driving tasks. As a result, drivers are lulled into states of mind where little attention is being devoted to the driving task and start to perform some other secondary tasks not connected with the driving per se, e.g., message texting, playing with infotainment, even internet browsing.

All the above said is well known in the art, so many technical solutions are directed to solve the observed problem by camera tracking the driver's face/eyes to estimate the driver's gaze zone. The gaze zones, usually classified by importance, are correlated and combined with the vehicle behavior and an algorithm assesses the driver's attention directed to driving. It is common in the art that such algorithms use a kind of driver's gaze scoring into the zone relevant to driving, the zone relevant to vehicle instrumentation, and other non-relevant zones. Such scoring is performed and averaged over one or more time windows, from which is then deduced the current driver's state, i.e., the level of distraction.

However, it seems that the interplay between the driver's gaze zones and the influence of possible driver's gaze choices in future times are not calculated in the models used in the art. Furthermore, the constant looking in the zone relevant, or partially relevant, to driving may also be a problem. A technical problem (among one or more technical problems) solved by one or more embodiments the present disclosure can relate to an improvement in a scoring scheme used in a visual distraction detection method or system in the driving monitoring system. The scoring scheme according to one or more embodiments of the present disclosure can introduce the correlations among the zones and the possibility that the zone's classification is changed based on its purpose in current driving conditions and its relevance in the scoring scheme by the recorded gaze history and possible future gazes. Furthermore, one or more embodiments of the present disclosure can implement the concept of potential usefulness level (PUL) for all zones in the scoring process for some given frame, assigning the potential dynamics, i.e., zone involvements, for all zones. For all secondary and tertiary zones, i.e., the gaze zones which are not necessary for keeping the vehicle trajectory, their PUL values decrease in time and henceforth their scoring contribution in the scoring scheme. The PUL values of other gaze zones, that are not detected in the current frame, can be potentially modified if they are correlated with the gaze zone detected in the current frame and combined with the vehicle status.

The scoring enhancements according to one or more embodiments of the present disclosure, based on the PUL values, can produce better results in detecting secondary tasks. Also, a novel 4-level scoring scheme can be provided or implemented for providing one or more outputs such as for alerting the driver, automatically controlling the vehicle, etc.

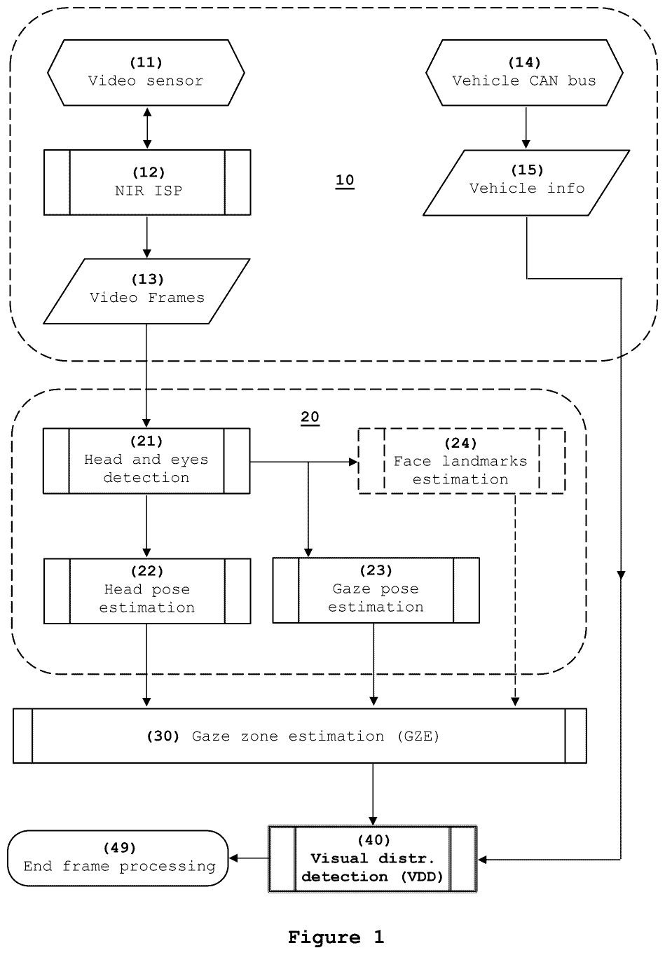

A driving monitoring system (DMS) according to one or more embodiments of the present disclosure is depicted in FIG. 1. The system may comprise or consists of an external data feed (10), and a video frame processor module (20) which can result in a gaze zone estimation value (30) for each captured frame. The gaze zone estimation value (30) together with the vehicle info (15), being part of the external data feed (10), can be further processed in a visual distraction detection (VDD) module (40), which may be regarded as a visual distraction detector (VDD), which can result in a multi-level output (e.g., 4-level output {0, 1, 2, 3}) VD value, upon which the alarm can be triggered, and the end frame processing (49) occurs. The visual distraction detection (VDD) module can produce different outputs and processes the input information differently. Some or all of the DMS can be implemented in or using circuitry. For instance, some or all of the modules can be implemented in or using circuitry.

### Vehicle Zones

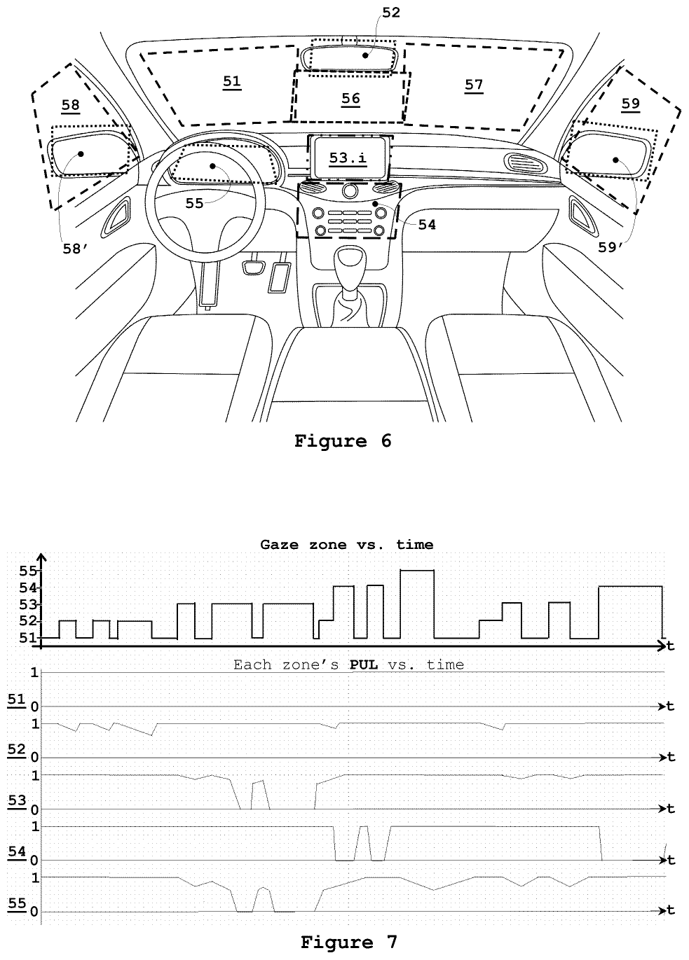

FIG. 6 represents an example of a vehicle's interior divided into exemplary zones. Such division can be performed for each vehicle model separately. According to one or more embodiments, the zones can be the left windshield part (51), rearview mirror (52), the command console (54), the instrument cluster (55), the central windshield part (56), the right windshield part (57), the left window (58), the left side mirror (58′), the right window (59), and/or the right side mirror (59′). The instrument cluster (55) can be, generally speaking, an instrument table with a set of signals (speed, RPM, lights, fuel level, . . . ) that can be implemented for driving. Left/right side mirrors (58′, 59′) may be visible only through the corresponding left/right windows (58, 59), and can represent the specific subset of driver's gazes directed to the areas denoted as (58, 59). The distinction from zones (58, 58′) or (59, 59′) can be extracted also by vehicle data (15), for instance, if the adequate turn signal is on, there can be a high probability that the corresponding side mirror is used by the driver.

Furthermore, there can be Central Information Display (CID) (53.i), which can be a dynamically changed zone, where i=1, 2, . . . , n defines various screen contents with different impacts on the driver's attention. The navigation pane is represented with (53.1), the radio/audio set with (53.2), the air condition settings (53.3), the vehicle set-up (54), etc., though embodiments of the present disclosure are not limited to those specifically shown. For driving purposes, looking into (53.1), can help the driver, and such a gaze can be classified differently than the gaze directed in the same direction, but on a different content such as zone (53.2, 53.3).

### External Data Feed

Different kinds of data (e.g., two kinds, only two kinds, etc.) can be implemented for the smooth operation of the driving monitoring system (DMS) such as depicted in FIG. 1, vehicle and video data.

One or more cameras directed toward the driver's face can be used, and camera positions may be mounted in the zones (55, 56) and/or in the pillars that separate zones (51, 58) and/or zones (57, 59). Close to each camera may be situated one or more near-infrared (NIR) light sources, usually operating on 940 nm, but other variants using 850 nm may also be implemented. NIR can allow the DMS to work at night and/or to penetrate glasses or sunglasses without attenuation (e.g., unsuitable attenuation, major attenuation, etc.). Each camera's video sensor (11) can capture many frames per second (fps), usually 10-60 fps which can be processed by the near-infrared image signal processing (ISP) module (12). A role of the module (12), among one or more roles, can be to regulate the brightness of the captured frame within the range which allows further processing. Once the captured frame is normalized, it can be regarded as ready to be sent to the gaze estimator (20).

Vehicles according to embodiments of the present disclosure can implement a Controller Area Network (CAN) bus, or similar network, that integrates vehicle systems (14) regarding the data exchange among them. Such vehicle systems can exchange data in real-time. CAN bus can be regarded as standardized and can allow easy access to all necessary vehicle info (15) for operating the DMS. Vehicle info (15), retrieved by the DMS, can comprise data regarding the speed, the steering angle, and/or the turn signal, as examples. Optionally, the vehicle info (15) can additionally or alternatively comprises the CID status (53.i), which can be dynamically changed in course of driving and can influence the decision logic of one or more of the visual distraction detection (VDD) module, distance from the vehicle ahead, illumination data such as night/day/tunnel, speed limits, weather conditions such as temperature, rain, or ice, etc.

### Gaze Estimator

The gaze estimator (20) can be comprised or can consist of a plurality modules (some or all of which may be implemented in or using circuitry). The head and eyes detection module (21) can extract the driver's head and subsequently the eyes in each frame by using, for instance, deep neural networks (DNN) trained for the task or any other suitable approach. The data from module (21) can be fed to the head pose estimation module (22), the gaze poses estimation module (23), and/or to the face landmarks estimation module (24). Again, it can be possible to use the DNN approach to extract the driver's head pose from the module (22) and based on the head and eyes poses to define the driver's gaze pose estimation. Having in mind the prior vehicle's interior zones definition, the outcome from the gaze estimator (20) can be the driver's gaze zone estimation value (30) for the current frame, for instance, the value which indicates that the driver is looking in the left windshield part (51) in this processed frame.

The approaches may differ in the level of certainty depending on the used calculation/estimation model(s) used for the gaze extraction methods.

### Visual Distraction Detection (VDD) Module

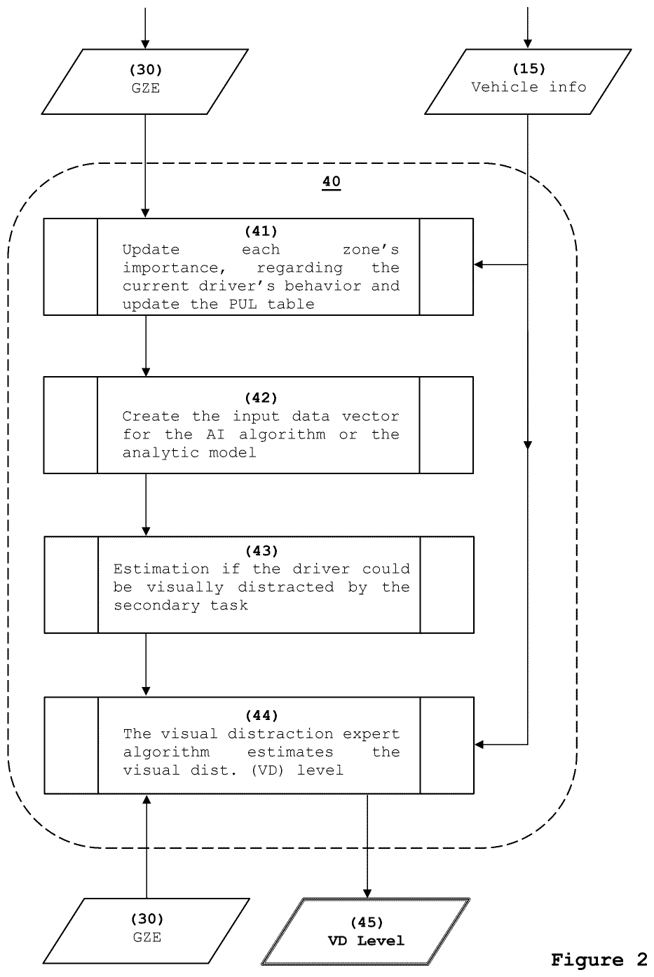

A VDD module (40) according to one or more embodiments is depicted in FIG. 2. The gaze zone estimation value (30) and vehicle info (15) can be fed to the VDD module in several processing stages. Some or all of the VDD module (40) can be implemented in or using circuitry.

The VDD module can implement a scoring method or methodology, which can be performed over a plurality time windows and for each newly received frame. Time windows can be chosen that last between 2-45 seconds, as an example. According to one or more embodiments, the time window can last between 2-12 seconds and if one uses a set of time windows that lasts {e.g., 2 sec, 4 sec, 6 sec., 8 sec, 10 sec., 12 sec.} where the time window duration can be a kind of integration factor in the performed scoring. The number of frames analyzed by the VDD module can depend on the current fps rate defined by the camera or by the gaze zone estimation (GZE) output. Of course, the different approaches are also feasible.

The scoring process can produce a value between 0 and 1 for each time window accompanied by the driver's gaze zone usage distribution within the primary, secondary, and tertiary gaze zone for each selected time window.

According to one or more embodiments, the zones may not be equal with respect to driver distraction. For the scoring purpose, the estimated driver's gaze zone can be classified in the processing of each newly received frame as:
primary gaze zone, if it serves as the gaze zone for keeping the vehicle trajectory,
secondary gaze zone if it serves for planning the driving, and
tertiary gaze zone for all other gaze zones.

The above classification can be performed in the zone updating module (41), which can be comprised of or consist of other modules as well.

The scoring for each time window can be calculated by averaging scores of all captured frames within the said time window, where the score for each captured frame is its potential usefulness level (PUL) value, ranging from 0-1. This PUL value can be retrieved from the PUL table, assigned to the vehicle zone, which can be recorded the driver's gaze zone estimation (30) in the current frame.

The potential usefulness level (PUL) value for some particular zone (51, . . . , 59) may depend on the involvement of the zone in a particular segment of the driving process, as well as the corresponding increasing or decreasing rate for the zone. According to one or more embodiments:
(i) for the gaze zone detected in the current frame as the primary gaze zone its PUL value is set to 1,
(ii) for the gaze zone detected in the current frame as the secondary or tertiary gaze zone, its PUL value is decreased, and
(iii) the PUL values of other gaze zones, that are not detected in the current frame, are potentially modified if they are correlated with the gaze zone detected in the current frame.

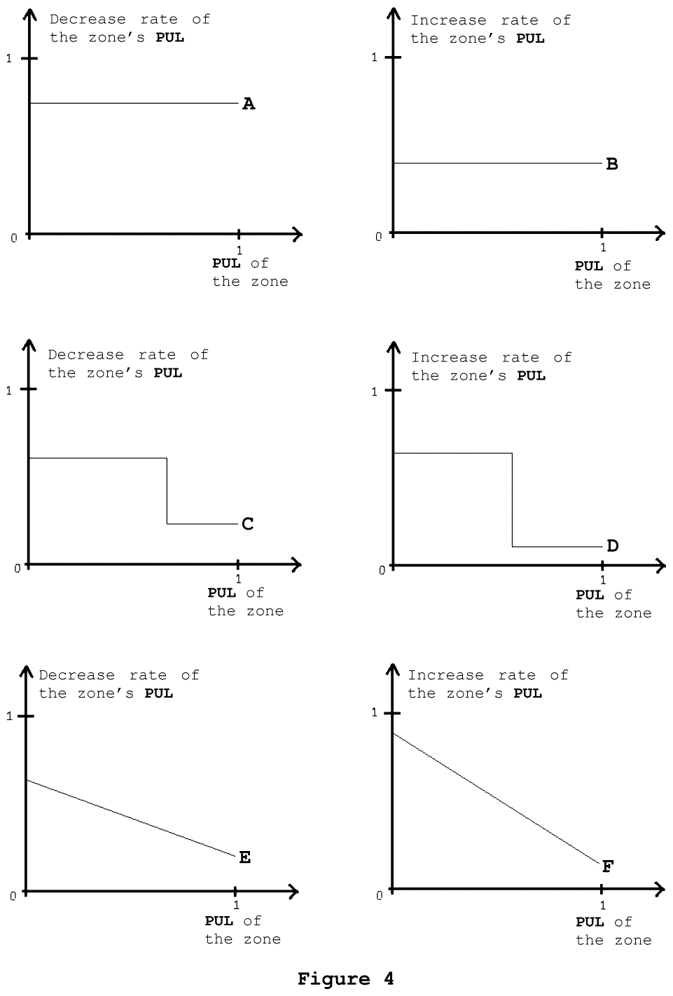

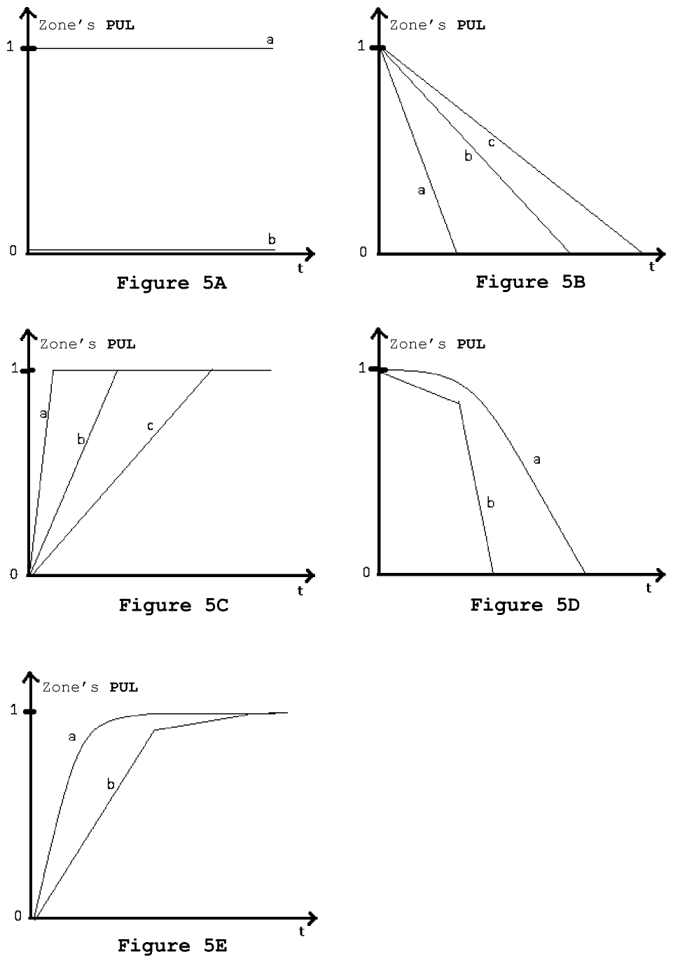

To illustrate the above, we consider, as but one example, a lane change from the right to the left line on a highway, for the left-seated driver. The primary zone can be certainly the zone (51) depicted in FIG. 6, while the zone helping in driving planning can be the zones (58′, 52) by which the driver should monitor the lane change allowance. So, at the beginning of the line-changing action, the secondary zones can be useful, and the PUL values can be high, such as depicted in FIG. 5B, with different slopes for each zone (58′, 52). Continuous looking into these zones can mean that the driver is not concentrated on the primary zone (51), so the PUL value of the zones can rapidly decrease in time, with, for instance, the constant decrease rate, such as depicted in FIG. 4, case A. The PUL values for zones (58′, 52) therefore can decrease from “very useful” at the beginning of line changing action where the PUL value is set to 1, to “not useful” after 2-3 seconds of constant looking into the zones, where the PUL value is 0.

FIG. 4, cases A-F, depicts various increasing/decreasing rates depending on the current PUL value for some particular zone. For instance, in case C where the decrease rate value has a kink, corresponds to the time dependency of the same zone as depicted in FIG. 5D, case “b”—kinked decreasing slope in time. Similarly, if the PUL increase rate is as depicted in FIG. 4, case F, the PUL value time behavior for the same zone is depicted in FIG. 5E, graph denoted by “a”. FIGS. 5A-5E are examples of exemplary models for increasing/decreasing the PUL values of one or more of the zones.

Furthermore, note the interdependencies of secondary zones and their contribution. Earlier, it was mentioned that the PUL values of other zones, that are not detected in the current frame, can be potentially modified if they are correlated with the gaze zone detected in the current frame. In the previously discussed lane-changing action, the secondary zones, such as the expressly mentioned secondary zones, i.e., the left rear window (58′) and the rearview mirror (52) can be interconnected. Gazing into the left rear window (58′) can modify the PUL value of another secondary zone (52) by decreasing (e.g., drastically) its value for the future frame, because it can mean that the driver is not paying attention to the primary zone.

Interlinked, i.e., correlated zones via the PUL values and their corresponding increase or decrease rates with the examples depicted in FIG. 4, cases A-F, can produce some or all effects depicted in FIGS. 5A-5D. Once again, it is worth noting that the PUL level can be maintained within the range [0,1].

FIG. 7 represents 5-zones interplay. The involved zones, according to FIG. 6, can be the left windshield part (51), the rearview mirror (52), the CID having the navigation pane selected (53), the command console (54), and the instrument cluster (55). The upper graph in FIG. 7 represents the gaze zones' estimation performed vs. time. Further graphs represent the PUL value change over time, for each zone. For the primary gaze zone (51), the PUL value can be 1 (e.g., always 1). The rearview mirror (52) PUL value can start to decrease each time the driver looks into the zone and can increase each time the driver looks in the primary gaze zone. An example of another zone relationship interplay can be visible when the driver uses CID (53) for navigation and instrument cluster (55) zones. In the given example, when the driver looks at the navigation (53), the PUL of the mentioned zone can decrease over time, and, at the same time, the PUL of the instrument cluster zone (55) can decrease also. It is worth noticing that the PUL decrease rate functions of both zones can be different. On the other hand, when the driver's gaze is directed to zone 55, there may be no negative effect on zone 53 PUL.

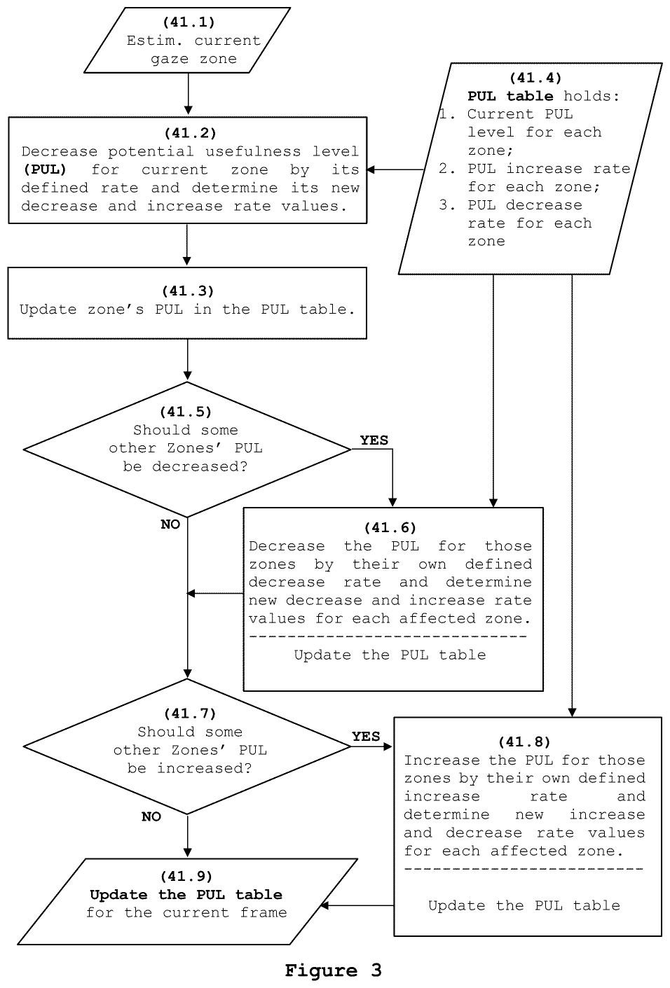

FIG. 3 depicts schematically the PUL table update process for non-primary gaze zones, and over each frame processing, according to one or more embodiments of the present disclosure. Once the current gaze zone data (41.1) enters the module, the potential usefulness level (PUL) for the current gaze zone can start to decrease by its defined rate and its new decrease and increase rate values can be determined, step (41.2). Then, the PUL table can be updated in step (41.3) with newly selected values according to the user model. If some other zones are affected by the currently processed gaze zone, then in step (41.5) and in step (41.7) those affected zones, i.e., their increase and decrease rates can be updated as well as their PUL values, modules (41.6, 41.8). An outcome from module (41), which may be regarded as the final output or outcome from the module (41), can be a fully updated PUL table for the current frame.

Now, the VDD module (40), more particularly module (41) can be configured to calculate a value between 0 and 1 for each time window accompanied by the driver's gaze zone usage distribution within the primary, secondary, and/or tertiary gaze zone for each selected time window of different pre-selected durations. These data can be used to create a data vector that can consist of or can be comprised of at least primary, secondary, and/or tertiary gaze zone usage distribution and corresponding scoring data performed for several predefined time windows in the module (42). The VDD module (40) and/or the module (41) can be implemented in or using circuitry.

As the next step, the driver's mental focus on potential secondary tasks can be extracted, which can be performed in the module (43). To perform such estimation, a trained AI algorithm can be used, or an equivalent analytical expression. In practice, the input vectors can be used for training the AI algorithm capturing the input vectors for selected windows, and assigning various secondary tasks with it. Examples of the selected secondary tasks can be cellular phone usage such as texting and internet browsing, playing with the infotainment, chatting with the co-driver, eating, etc. All these tests can be performed in controlled environments, such as polygons for driving, runways, etc, not compromising the test drivers' safety and allowing them to push their secondary tasks to a maximum resulting, sometimes, in a loss of vehicle's control. The comparison among the AI algorithm and the analytical expressions using thresholds can be tested and approximately 15 percent better results in reliable detection of the secondary driver's tasks can be performed with the AI algorithm in the form of SVM. So, the module (43) can extract the driver's mental focus and signals with TRUE if the driver is involved in any detectable secondary task, by the specified model. The module (43) can be implemented in or using circuitry.

All data from previous steps can be used in the module (44) to estimate the driver's visual distraction level (VDL) which can result in the discrete output {0, 1, 2, 3} and optionally activating the output (e.g., alarm, autonomous or assisted driving. The estimation can be performed by a visual distraction expert algorithm which combines the data regarding the secondary tasks from the module (43), the vehicle info (15), and the instant gaze zone estimation (30) for the current frame. The working principle of the visual distraction expert algorithm, contained in module (44), is explained below. The module (44) can be implemented in or using circuitry.

The VD level can be set to 1 (e.g., immediately) when the module (43) detected a secondary task performed by the driver. According to one or more embodiments, the detected secondary task can be related (e.g., closely) to the vehicle speed. So, the VD level can be raised to 2 and can trigger one or more outputs (e.g., one or more alarms) if the speed is above the lower threshold speed, and optionally accompanied by finishing a search for a parking slot or similar look-around action, and
(i) for speeds higher than the upper threshold speed, the output(s) (e.g., alarm) can be activated the moment after the level of visual distraction is at level 1, or
(ii) for speeds between the lower threshold speed and the upper threshold speed, the output(s) (e.g. alarm) can be activated within the delay, depending on the secondary task intensity which can be calculated as a percentage of the TRUE state within the specified time window.

According to one or more embodiments, the lower threshold speed can be set to be 10 km/h, for instance, and the higher threshold speed can be set to be 60 km/h, for instance, but modifications depending on geographical locations, current legislation, or vehicle type can also be possible.

A source of accidents may occur in those cases where the driver's attention is attracted to some situation in one or more tertiary gaze zones. This can be solved by the module (44) in a way that the duration of the current gaze is evaluated, and if the current gaze is directed in the tertiary gaze zone for a time greater than the defined time interval, the VD level can be raised to 2, for the following conditions:
(i) for speeds that are lower than the lower threshold speed, the defined time interval can be Thi,
(ii) for speeds between the lower threshold speed and the upper threshold speed, the defined time interval can be decreased from Thi to Tlow (e.g., linearly), and
(iii) for speeds above the upper threshold speed, the defined time interval can be set to Tlow;
where the time interval Thi can be greater than Tlow time interval. As but one example, suitable results (e.g., the best) can be achieved with Thi set to 2.5 sec and Tlow to 2.0 sec.

Furthermore, if the current gaze is not directed in the primary gaze zone for the predefined time interval, e.g., 3-6 sec, preferably 4 sec, the VD level can be raised to 2. Also, the VD level can be set to 3 if the VD level is maintained on 2 for the time interval, e.g., 0.5-5 sec, preferably 1-3 sec, where the time interval is selected to be inversely proportional to the vehicle speed, that to say, the interval can be shorter if the speed is greater. Finally, some or all output(s) (e.g., alarms) can be turned off or suspended if the driver's gaze is directed to the primary gaze zone longer than the predefined time interval. This can be achieved, for instance, by the recovery signal reaching the value 1.

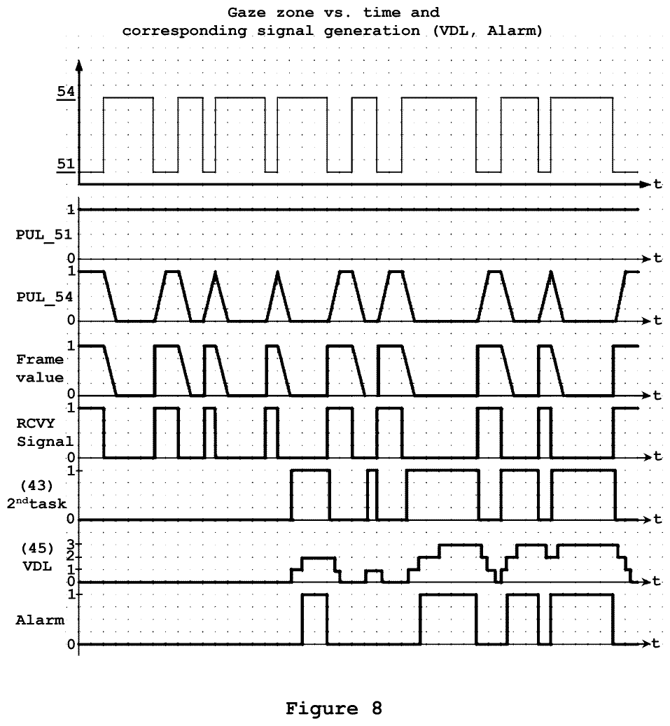

It is instructive to examine FIG. 8 which illustrates the above in an artificial example where the gaze zone is changed from the left windshield part (51) to the command console (54), as depicted in FIG. 6. The top graph indicates gaze zone variations in time. Zone (51) can be the primary zone, so the corresponding PUL_51 value can be set to 1 (e.g., always set to 1). The command console (54) can be the tertiary zone with the assigned steep decrease and increase the rate of its PUL_54 value, which may be oscillating from 0 to 1 and vice versa, triggered by the gaze switch between these two zones (54, 51), for instance. Frame score can be the assigned score for the extracted frame in any time instance, i.e., the instant PUL value. RCVY signal can be a recovery signal that can (e.g., basically) correspond with the engagement of the primary zone (51), when the primary gaze is detected, the RCVY signal can be set to 1. FIG. 8 can be regarded as showing the driver's secondary task (2^{nd} task) involvement, generating the VDL (visual distraction level) raising, firstly to 2 and subsequently to 3, being correlated with intensive gazing to the command console (54). As an example, the bottom signal can be an alarm signal turned on (value=1) or off (value=0).

In the case of an alarm signal as one or more of the outputs, such alarm signal can be selected from one or more signals from the group consisting of or comprised of visual alarms, audio alarms, automatic braking, shaking the steering wheel, and/or turning on/off different devices in the vehicle cabin. Optionally, such a signal can be transmitted and processed with one or more other vehicle driver safety systems.

### Error Trapping

Error trapping may play a role in some processed captured frames. There may be situations when gaze estimation is not possible for various reasons, for instance, the driver's head is not detected correctly or the eyes remained closed, or someone hides one or more cameras by hand or other objects. There are ways to bridge the observed situation. For instance, the VDD module can treat such a situation as the gaze directed into the primary gaze zone. Another possibility is to use linear interpolation over the missing frames and to “reconstruct” the observed data gaps.

### CONCLUSION

The examples discussed hereby serve only to exemplify the potential of the visual distraction detection method and system. According to one or more embodiments, the first time the potential usefulness level (PUL) of the driver's gaze zones, which may not be currently detected from the gaze estimation model, can be used in detecting potential secondary drivers' tasks and performing suitable scoring by capturing the driving past and the driving future, i.e., using of the zones that might contribute to driver's attention.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

The functionality of the elements disclosed herein may be implemented using circuitry or processing circuitry which includes general purpose processors, special purpose processors, integrated circuits, ASICs (“Application Specific Integrated Circuits”), conventional circuitry and/or combinations thereof which are configured or programmed to perform the disclosed functionality. Processors are considered processing circuitry or circuitry as they include transistors and other circuitry therein. The processor may be a programmed processor which executes a program stored in a memory. In the disclosure, the circuitry, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

Further, as used herein, the term “circuitry” can refer to any or all of the following: (a) hardware-only circuit implementations (such as implementations in only analog and/or digital circuitry); (b) to combinations of circuits and software (and/or firmware), such as (as applicable): (i) a combination of processor(s) or (ii) portions of processor(s)/software (including digital signal processor(s)), software and memory(ies) that work together to cause an apparatus, such as a mobile phone or server, to perform various functions); and (c) to circuits, such as a microprocessor(s) or a portion of a microprocessor(s), that require software or firmware for operation, even if the software or firmware is not physically present. This definition of “circuitry” can apply to all uses of this term in this application, including in any claims. As a further example, as used in this application, the term “circuitry” can also cover an implementation of merely a processor (or multiple processors) or portion of a processor and its (or their) accompanying software and/or firmware.

Use of the terms “data,” “content,” “information” and similar terms may be used interchangeably, according to some example embodiments of the present disclosure, to refer to data capable of being transmitted, received, operated on, and/or stored. The term “network” may refer to a group of interconnected computers or other computing devices. Within a network, these computers or other computing devices may be interconnected directly or indirectly by various means including via one or more switches, routers, gateways, access points or the like.

Aspects of the present disclosure have been described above with reference to flowchart illustrations and/or block diagrams of methods, apparatus (systems) and computer program products according to embodiments of the present disclosure. In this regard, the flowchart and block diagrams in the Figures illustrate the architecture, functionality, and operation of possible implementations of systems, methods and computer program products according to various embodiments of the present disclosure. For instance, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### INDUSTRIAL APPLICABILITY

The industrial application of this approach in constructing a visual distraction detection (VDD) method in a driving monitoring system (DMS) is obvious. The said method belongs to the field of image or video recognition or understanding, used inside the vehicle, that is designed for recognizing the driver's state of behavior. The method belongs to the field of image or video recognition or understanding, used inside the vehicle, that is designed for recognizing the driver's state of behavior, e.g., attention or drowsiness. In addition, the disclosed system sends the signals, based on the estimation or calculation in relation to the driver, to other vehicle systems to warn the driver or to execute the set of safety actions.

### REFERENCE NUMBERS ASSOCIATED WITH EXAMPLE OR EXEMPLARY FEATURES

10 External data feed
11 Video sensor
12 Near-infrared (NIR) image signal processing (ISP)
13 Video frames
14 Vehicle system, e.g., Controller Area Network (CAN) bus
15 Vehicle info
20 Gaze estimator
21 Head and eyes detection module
22 Head pose estimation module
23 Gaze pose estimation module
24 Face landmarks estimation module
30 Gaze zone estimation (GZE), within the vehicle
40 Visual distraction detector/detection module (VDD)
41 Zone updating module
41.1 Current gaze zone data
41.2 Decrease the potential usefulness level (PUL)
41.3 Update the PUL table for the current gaze zone
41.4 PUL table and increase/decrease rates
41.5 Decrease the PULs of other zones?
41.6 Decrease the PULs of other zones, and update rates.
41.7 Increase the PULs of other zones?
41.8 Increase the PULs of other zones, and update rates.
41.9 Iterated PUL Table
42 Module for creating data vectors, e.g., for AI algorithm
43 Module searching for secondary tasks
44 Module containing visual distraction expert algorithm
45 Visual Distraction Level (VDL)
49 End frame processing
51 Left windshield part
52 Rearview mirror
53.i Central Information Display (CID), e.g., dynamically changed zone, where i=1, 2, . . . , n defining various screen contents
54 Command console
55 Instrument cluster
56 Central windshield part
57 Right windshield part
58 Left window
58′ Left side mirror
59 Right window
59′ Right side mirror

1. A method of operation of a visual distraction detector, for use within a driving monitoring system that provides the visual distraction detector with the following data:
a driver's gaze zone estimation data for each captured frame by one or more cameras situated within the vehicle oriented toward the driver, where the gaze zones are previously defined for the vehicle with the provision that one or more of the gaze zones change their function in time, and where each of the gaze zones is dynamically classified for each recorded frame into primary, secondary, or tertiary gaze zone based on its purpose in current driving conditions, and
vehicle info, captured from one or more vehicle systems, which at least comprises data regarding speed, steering angle, and turn signal,
where the method of operation comprises the following for each newly captured frame:
A. performing the scoring of a plurality time windows by a value ranging from 0 to 1 and calculating the driver's gaze zones distribution within the primary, secondary, and tertiary gaze zones for each selected time window,
where at least one time window of the plurality of time windows lasts between 2-12 seconds, and where each at least one time window is sliced in subintervals defined by time width of captured camera frames,
where the scoring for each at least one time window is calculated by averaging scores of all captured camera frames within the time window, where the score for each captured camera frame is its potential usefulness level (PUL) value, retrieved from a PUL table, assigned to a vehicle zone to which is recorded the driver's gaze zone estimation for the current camera frame,
B. creating a data vector that of primary, secondary, and/or tertiary gaze zone usage distribution and corresponding scoring data performed for predefined time windows,
C. extracting the driver's mental focus and signals with TRUE under a condition where the driver is involved in any detectable secondary task by using the data from step B and performing the estimation via a previously trained AI algorithm or using an analytical expression resulting in the TRUE/FALSE output,
D. estimating the instant driver's visual distraction level (VDL) resulting in the discrete output {0, 1, 2, 3} and activating the alarm, where the estimation is performed by a visual distraction expert algorithm which combines the data from step C, vehicle information, and the instant gaze zone estimation for the current frame,
wherein the gaze zones are re-classified for each captured camera frame such that the estimated driver's gaze zone is assigned as:
primary gaze zone, under a condition where it serves as the gaze zone for keeping the vehicle trajectory,
secondary gaze zone under a condition where it serves for planning the driving, and
tertiary gaze zone for all other gaze zones, and
where the PUL table for all gaze zones is updated for each frame such that:
(i) for the gaze zone detected in the current frame as the primary zone its PUL value is set to 1,
(ii) for the gaze zone detected in the current frame as the secondary or tertiary zone, its PUL value is decreased, and
(iii) the PUL values of other gaze zones, that are not detected in the current frame, are modified under a condition where they are correlated with the gaze zone detected in the current frame, and
wherein all new PUL values stay within the interval [0,1] and are stored in the PUL table together with their increase and decrease rates defined by the scoring model in step A.

2. The method of operation according to claim 1, wherein the PUL values of other gaze zones, that are not detected in the current frame, are currently increased, decreased, or remain unaffected, which depends on the contribution of each particular zone to the driver's visual distraction under a condition where, in the next frame, this zone is estimated as the driver's gaze zone.

3. The method of operation according to claim 1, wherein the PUL decrease or increase rate for any gaze zone is a unique function of time, the zone, its currently recorded PUL value, and predefined interaction mode with other potential gaze zones.

4. The method of operation according to claim 1, wherein the VD level is set to 1 under a condition where step C detected a secondary task performed by the driver.

5. The method of operation according to claim 4, wherein the VD level is raised to 2 and triggers the alarm under a condition where the speed is above the lower threshold speed, and
(i) for speeds higher than the upper threshold speed, the alarm is activated responsive to the level of visual distraction being set at level 1, or
(ii) for speeds between the lower threshold speed and the upper threshold speed, the alarm is activated within a delay, depending on the secondary task intensity which is calculated as a percentage of the TRUE state within the specified time window.

6. The method of operation according to claim 1, wherein the duration of the current gaze is evaluated, and under a condition where the current gaze is directed in the tertiary zone for a time greater than the defined time interval, the VD level is raised to 2, for the following conditions:
(i) for speeds that are lower than the lower threshold speed, the said defined time interval is Thi,
(ii) for speeds between the lower threshold speed and the upper threshold speed, the said defined time interval is linearly decreased from Thi to Tlow, and
(iii) for speeds above the upper threshold speed, the said defined time interval is set to Tlow;
where the time interval Thi is greater than Tlow time interval.

7. The method of operation according to claim 5, wherein the lower threshold speed is set to be 10 km/h, the higher threshold speed is set to be 60 km/h, while Thi is set to 2.5 sec and Tlow to 2.0 sec.

8. The method of operation according to claim 6, wherein the lower threshold speed is set to be 10 km/h, the higher threshold speed is set to be 60 km/h, while Thi is set to 2.5 sec and Tlow to 2.0 sec.

9. The method of operation according to claim 1, wherein the duration of the current gaze is evaluated, under a condition where the current gaze is not directed in the primary zone for the predefined time interval the VD level is raised to 2.

10. The method of operation according to claim 4, wherein the VD level is set to 3 under a condition where the VD level is maintained on 2 for the time interval 0.5-5 sec, where the time interval is inversely proportional to the vehicle speed.

11. The method of operation according to claim 8, wherein the VD level is set to 3 under a condition where the VD level is maintained on 2 for the time interval 0.5-5 sec, where the time interval is inversely proportional to the vehicle speed.

12. The method of operation according to claim 1, where the alarm is selected from one or more signals from the group consisting of visual alarms, audio alarms, automatic braking, shaking a steering wheel, and turning on/off different devices in the vehicle cabin, and sends a signal to other vehicle driver safety systems.

13. The method of operation according to claim 1, wherein all alarms are turned off or suspended under a condition where the driver's gaze is directed to the primary gaze zone longer than the predefined time interval.

14. A driving monitoring system comprising:
circuitry configured to perform a method of operation of a visual distraction detector, based on or using the following data:
a driver's gaze zone estimation data for each captured frame by one or more cameras situated within the vehicle oriented toward the driver, where the gaze zones are previously defined for the vehicle with the provision that one or more of the gaze zones change their function in time, and where each of the gaze zones is dynamically classified for each recorded frame into primary, secondary, or tertiary gaze zone based on its purpose in current driving conditions, and
vehicle info, captured from one or more vehicle systems, which at least comprises data regarding speed, steering angle, and turn signal,
where the method of operation comprises the following for each newly captured frame:
A. performing the scoring of a plurality time windows by a value ranging from 0 to 1 and calculating the driver's gaze zones distribution within the primary, secondary, and tertiary gaze zones for each selected time window,
where at least one time window of the plurality of time windows lasts between 2-12 seconds, and where each at least one time window is sliced in subintervals defined by time width of captured camera frames,
where the scoring for each at least one time window is calculated by averaging scores of all captured camera frames within the time window, where the score for each captured camera frame is its potential usefulness level (PUL) value, retrieved from a PUL table, assigned to a vehicle zone to which is recorded the driver's gaze zone estimation for the current camera frame,
E. creating a data vector that of primary, secondary, and/or tertiary gaze zone usage distribution and corresponding scoring data performed for predefined time windows,
F. extracting the driver's mental focus and signals with TRUE under a condition where the driver is involved in any detectable secondary task by using the data from step B and performing the estimation via a previously trained AI algorithm or using an analytical expression resulting in the TRUE/FALSE output,
G. estimating the instant driver's visual distraction level (VDL) resulting in the discrete output {0, 1, 2, 3} and activating the alarm, where the estimation is performed by a visual distraction expert algorithm which combines the data from step C, vehicle information, and the instant gaze zone estimation for the current frame,
wherein the gaze zones are re-classified for each captured camera frame such that the estimated driver's gaze zone is assigned as:
primary gaze zone, under a condition where it serves as the gaze zone for keeping the vehicle trajectory,
secondary gaze zone under a condition where it serves for planning the driving, and
tertiary gaze zone for all other gaze zones, and
where the PUL table for all gaze zones is updated for each frame such that:
(iv) for the gaze zone detected in the current frame as the primary zone its PUL value is set to 1,
(v) for the gaze zone detected in the current frame as the secondary or tertiary zone, its PUL value is decreased, and
(vi) the PUL values of other gaze zones, that are not detected in the current frame, are modified under a condition where they are correlated with the gaze zone detected in the current frame, and
wherein all new PUL values stay within the interval [0,1] and are stored in the PUL table together with their increase and decrease rates defined by the scoring model in step A.

15. The system according to claim 14, wherein the PUL values of other gaze zones, that are not detected in the current frame, are currently increased, decreased, or remain unaffected, which depends on the contribution of each particular zone to the driver's visual distraction under a condition where, in the next frame, this zone is estimated as the driver's gaze zone.

16. The system according to claim 14, wherein the PUL decrease or increase rate for any gaze zone is a unique function of time, the zone, its currently recorded PUL value, and predefined interaction mode with other potential gaze zones.

17. The system according to claim 14, wherein the VD level is set to 1 under a condition where step C detected a secondary task performed by the driver.

18. The system according to claim 14, wherein the duration of the current gaze is evaluated, and under a condition where the current gaze is directed in the tertiary zone for a time greater than the defined time interval, the VD level is raised to 2, for the following conditions:
(iv) for speeds that are lower than the lower threshold speed, the said defined time interval is Thi,
(v) for speeds between the lower threshold speed and the upper threshold speed, the said defined time interval is linearly decreased from Thi to Tlow, and
(vi) for speeds above the upper threshold speed, the said defined time interval is set to Tlow;
where the time interval Thi is greater than Tlow time interval.

19. The system according to claim 14,
wherein the duration of the current gaze is evaluated, under a condition where the current gaze is not directed in the primary zone for the predefined time interval the VD level is raised to 2, and
wherein the VD level is set to 3 under a condition where the VD level is maintained on 2 for the time interval 0.5-5 sec, where the time interval is inversely proportional to the vehicle speed.

20. The system according to claim 14, wherein all alarms are turned off or suspended under a condition where the driver's gaze is directed to the primary gaze zone longer than the predefined time interval.
